(19) United States
(12) Patent Application Publication
(10) Pub. No.: US 2010/0144004 A1
(43) Pub. Date: Jun. 10, 2010

(54) Cell culture system, cell culture substrate thereof and culture method thereof

(75) Inventors: Cheng Zhong, Beijing (CN); Li Fan, Beijing (CN)

Correspondence Address: PCE INDUSTRY, INC.;ATT. Steven Reiss, 288 SOUTH MAYO AVENUE, CITY OF INDUSTRY, CA, 91789, US

(73) Assignees: Tsinghua University, Beijing City (CN); HON HAI Precision Industry CO., LTD., Tu-Cheng City (TW)

(21) Appl. No.: 12/462,729

(22) Filed: Aug. 7, 2009

(30) Foreign Application Priority Data
Aug. 9, 2008 (CN) 200810142286.1

(51) Int. Cl. C12N 13/00; C12M 1/00; C12N 5/02

(52) U.S. Cl. 435/173.8; 435/289.1; 435/395; 977/742

(57) ABSTRACT

A cell culture system for directing growth of cells is provided. The cell culture system includes a substrate, a culture layer and a culture medium. The culture layer is located on the substrate. The culture layer includes at least one carbon nanotube film including a plurality of carbon nanotubes orientated primarily along a same direction. The culture medium covers the culture layer and includes at least one growth factor.

### BACKGROUND

1. Technical Field

The present disclosure relates to cell culture systems and substrates as well as culture methods, and particularly to a cell culture system, a cell culture substrate and a culture method for directing growth of cells.

2. Description of Related Art

The nervous system is a complex cellular communication network that is mainly composed of neurons and glial cells (neuroglial cells). Glial cells occupy spaces between neurons and modulate neurons' functions. The neurons sense features of both external and internal environments and transmit this information to the brain for processing and storage. For example, the neurons receive the diverse types of stimuli from the environment (e.g. light, touch, sound) and convert into electric signals, which are then converted into chemical signals to be passed on to other cells.

Neurons exist in a number of different shapes and sizes, and can be classified by their morphology and function. The basic morphology of neuron consists of a cell body and neurites projecting/branching from the cell body towards other neurons. The neurites also can be defined two types by their functions. One of them is commonly called “dendrites”, which branch around the cell body and are configured to receive signals from other neurons to cell body. The other one is commonly called “axon”, which branches from the cell body and grows continually without tapering. The axon is configured to conduct the signals away from the neuron's cell body. The end of axon has branching terminals that release neurotransmitter substances acting as chemical signals into a gap between the branching terminals and the dendrites of other neurons. Thus, the information or signal is propagated.

Once injury to the nervous system occurs, neuron damage will lead to neurite degeneration and retraction. If damage is severe, breaks in neurites of neuron are even presented. Consequentially, the signal transmission will be affected and the cellular communication with specific neurons is ceased. Generally, damage on the neurites will recover via a self-renewal mechanism. However, in most cases, because the neurites will grow in all directions, it is not easy to reconnect with the opposite terminals in broken neurites.

What is needed, therefore, is a cell culture system, a cell culture substrate and a culture method for guiding the growth of cells, aiding in damaged cells to recover efficiently.

### BRIEF DESCRIPTION OF THE DRAWINGS

Many aspects of the embodiments can be better understood with references to the following drawings. The components in the drawings are not necessarily drawn to scale, the emphasis instead being placed upon clearly illustrating the principles of the present cell culture system, cell culture substrate and culture method for directing the growth of cells.

FIG. 1 is a cross-sectional view of a cell culture system, in accordance with an exemplary embodiment.

FIG. 2 is a vertical view of the cell culture system of FIG. 1.

FIG. 3 is a schematic view of a carbon nanotube film, in accordance with an exemplary embodiment.

FIG. 4 is a flow chart of a culture method for directing the growth of cells, in accordance with an exemplary embodiment.

FIG. 5 is a schematic view of a culture method for directing the growth of cells, in accordance with the exemplary embodiment.

### DETAILED DESCRIPTION

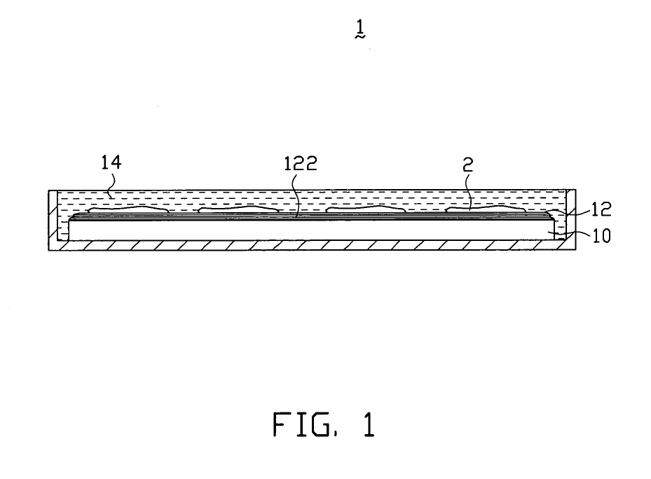

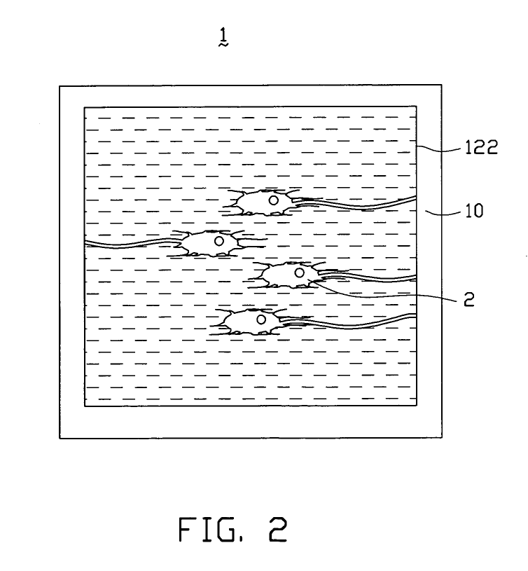

Referring to FIG. 1 and FIG. 2, one embodiment of a cell culture system 1 includes a substrate 10, a culture layer 12 and a culture medium 14. The culture layer 12 is located on the substrate 10. The culture medium 14 covers at least one portion of the culture layer 12. The neuronal cells 2 can be cultured in the cell culture system 1. The culture layer 12 can direct or guide neuronal cells 2 to grow in a specific direction. In one embodiment, the neuronal cells are hippocampal neurons.

The substrate 10 can be made of macromolecule. In the present embodiment, the substrate 10 can be a glass coverslip. Alternatively, the substrate 10 can be in a frame structure.

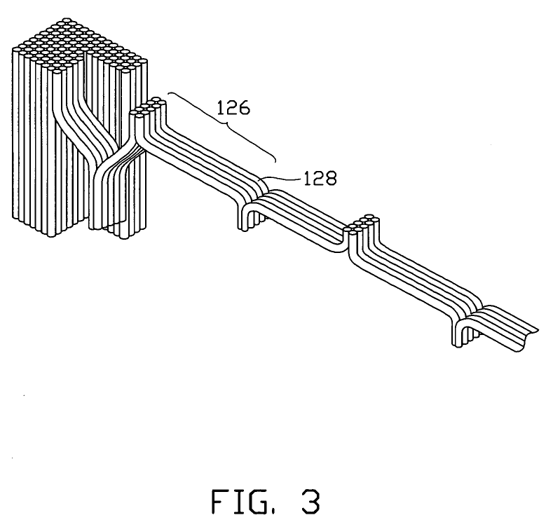

The culture layer 12 includes at least one carbon nanotube film 122, which is placed on the substrate 10. Referring to FIG. 3, the carbon nanotube film 122 includes a plurality of carbon nanotubes 128 orientated primarily the same direction. Particularly, the carbon nanotube film 122 can be fabricated by being drawn from a carbon nanotube array, which may be formed on a 4-inch silicon by vapor deposition. The carbon nanotube film 122 is comprised of a plurality of successively orientated carbon nanotube segments 126, which are joined end-to-end by van der Waals attractive force. Each carbon nanotube segment 126 is composed of a plurality of carbon nanotubes 128 substantially parallel to each other and with the same length.

Alternatively, the culture layer 12 can include two or more carbon nanotube films 122 that are stacked with each other on the substrate 10. In one embodiment, the culture layer 12 is formed by stacking about three to five carbon nanotube films 122 and has a thickness of about 10.0 nm to about 200.0 nm. In such case, the orientated directions of carbon nanotubes 128 in any two adjacent stacked carbon nanotube films 122 are parallel to each other.

The carbon nanotubes 128 of carbon nanotube film 122 can be multi-walled carbon nanotubes (MWCNTs). An average diameter of MWCNTs is in a range from about 10.0 nm to about 20.0 nm. Alternatively, the carbon nanotube film 122 comprising of single-walled carbon nanotubes (SWCNTs) also can be employed to culture neuronal cells. In one embodiment, MWCNTs are employed in the carbon nanotube film 122 and have an average diameter of about 15.0 nm.

In order to improve the hydrophilicity and biocompatibility of the carbon nanotube film 122, the carbon nanotubes 128 are functionalized to have pyrene group, anthracene group or carboxylic acid group by physical process or chemical process. Through attaching of appropriate functionalities onto the carbon nanotube scaffold, the derivational carbon nanotubes exhibit improved properties with respect to solubility and biocompatibility. In the present embodiment, the pyrene group and anthracene group are attached onto the surface of carbon nanotubes via physical absorption process. In addition, attachment of the carboxylic acid group (COOH) is achieved via chemical process.

The culture medium 14 covering the carbon nanotube film 122 includes at least one cell growth factor. In practice, the cell growth factor can be chosen according to types of cells 2 to be cultured. The cells 2, such as neuron cells, will grow and reproduce on the carbon nanotubes 128 and along the orientated directions of carbon nanotubes 128. In one embodiment, the cell growth factor can be a nerve growth factor (NGF) or a basic fibroblast growth factor (bFGF).

The cell culture system 1 of the present embodiment further can include a power supply, which is used to provide an electric signal to stimulate the neuronal cells 2. The stimulation treatment will facilitate the treated neuronal cells 2 to grow neuritis extending along the carbon nanotubes 128.

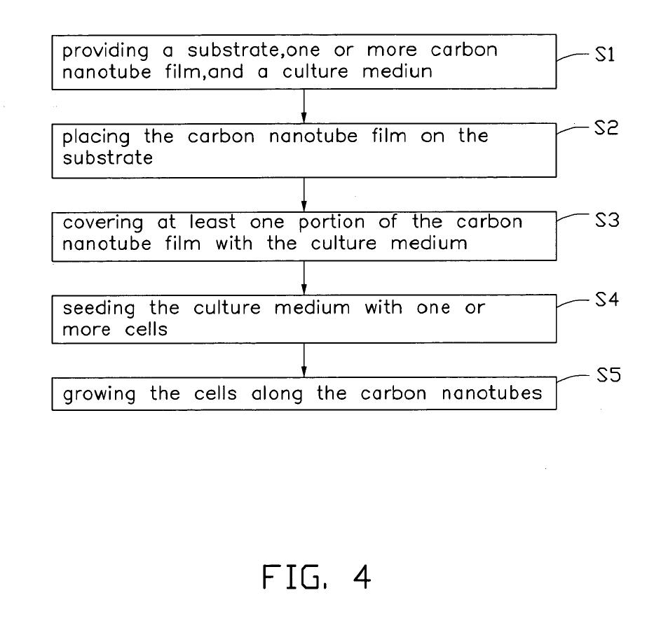

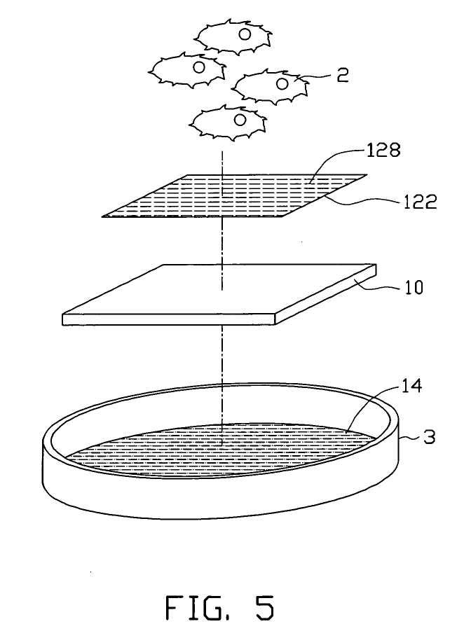

Referring to FIG. 3, FIG. 4 and FIG. 5, one embodiment of a culture method for directing the growth of cells includes providing a substrate 10, one or more carbon nanotube films 122 comprised of a plurality of carbon nanotubes 128, and a culture medium 14, S1. The carbon nanotube film 122 is placed on the substrate 10, S2. At least one portion of the carbon nanotube film 122 is covered with the culture medium 14, S3. One or more cells 2 are seeded in the culture medium 14, S4. The cells 2 grow along the carbon nanotubes 128, S5.

The method is described in more detail as follows.

In step S1, a substrate 10 serving as a supporting substrate is provided. The substrate 10 includes a base, which is made of macromolecule. The carbon nanotube films comprise of the carbon nanotubes orientated primarily along a same direction. In addition, the culture medium includes at least one growth factor. In the present embodiment, the carbon nanotube film 122 is fabricated by drawing from a carbon nanotube array (as shown in FIG. 3). The carbon nanotube film 122 can be formed by the following steps. A substrate, such as silicon substrate, is provided. A catalyst layer is deposited on the substrate by, for example, electron beam evaporation. The substrate with the catalyst layer is put into a reaction device. A mixture of a carbon source gas and a protecting gas is introduced into the reaction device. Thus, a carbon nanotube array is formed on the substrate. A group of carbon nanotubes 128 is pulled out from the formed carbon nanotube array by a tool, for example, tweezers. Thus, the carbon nanotube film 122 is formed by drawing out a plurality of carbon nanotubes 128 contiguously joined end to end by van der Waals attractive force therebetween. Particularly, the carbon nanotube film 122 is comprised of a plurality of carbon nanotube segments 126, each of which includes a plurality of carbon nanotubes 128 parallel to each other. The width of the carbon nanotube film can vary according to the practical needs.

Then, in step S2, the carbon nanotube film 122 is adhered on at least one surface of the substrate 10. The carbon nanotube film 122 can adhere to the substrate 10 by virtue of adhesive property of carbon nanotubes 128. In one embodiment, two or more carbon nanotube films 122 are provided and stacked with each other on the substrate 10. In such case, the orientated directions of carbon nanotubes 128 in different carbon nanotube films 122 are parallel to each other. The substrate 10 with the carbon nanotube films 122 is placed into a container 3.

In step S3, the culture medium 14 including at least one cell growth factor is added to the container 3 and cover the carbon nanotubes 128. In one embodiment, culture medium 14 is prepared by dissolving the cell growth factor in a biological media. In addition, the recipe of biological media can vary dependent on what type of cells 2 to be cultured.

In step S4, the cells 2 (e.g. neuronal cells) are seeded into the culture medium 14. The seeded cells 2 can attach onto the carbon nanotubes 128 of carbon nanotube film 122. In step S5, under the nourishment of biological media and the stimulation of the cell growth factor, the cells 2 are directed to grow along parallel arrangement of the carbon nanotubes 128.

In one embodiment, the culture method can further comprise a step of stimulating the cells 2 with an electric signal. The electric signal can be a current signal provided by a power supply. The cells 2 stimulated by current signal will speed the growth of neurites. In addition, the electric signal can pass or be transmitted through the culture medium to stimulate the cells 2. Alternatively, the electric signal can be directly applied to the carbon nanotube film 122.

Additionally, the culture method can further include a step of functionalizing the carbon nanotubes 128. Thus, the carbon nanotubes 128 can be modified to become more hydrophilic and biocompatible. In one embodiment, the carbon nanotubes 128 can be functionalized with pyrene group or anthracene group via physical absorption process. Alternatively, the carbon nanotubes 128 can be functionalized with carboxylic acid group via chemical process.

The carbon nanotube film of the embodiment provides a culture structure having a plurality of carbon nanotubes in linear form for the cells growing. As the carbon nanotubes are well arranged in a planar structure, the growth of cells is controllable to be guided or directed by the parallel arrangement of carbon nanotubes of carbon nanotube film. Thus, the cell culture system of the present embodiment can be employed to treat, e.g. damaged neuron cells, in particular to, neuron cells with damaged neurites. It is also understood that the apparatus and method disclosed herein is not limited to the growth of nerve cells, but can be employed to grow all suitable cells.

Finally, it is to be understood that the above-described embodiments are intended to illustrate rather than limit the disclosure. Variations may be made to the embodiments without departing from the spirit of the disclosure as claimed. The above-described embodiments illustrate the scope of the disclosure but do not restrict the scope of the disclosure.

It is also to be understood that above description and the claims drawn to a method may include some indication in reference to certain steps. However, the indication used is only to be viewed for identification purposes and not as a suggestion as to an order for the steps.

What is claimed is:

1. A cell culture system for directing growth of cells, comprising:
a substrate;
a culture layer located on the substrate, the culture layer comprising at least one carbon nanotube film, the carbon nanotube film comprising a plurality of carbon nanotubes orientated primarily along a same direction; and
a culture medium comprising at least one growth factor.

2. The cell culture system of claim 1, wherein the carbon nanotube film comprises of a plurality of successively orientated carbon nanotube segments, the carbon nanotube segments comprise of the plurality of carbon nanotubes.

3. The cell culture system of claim 1, wherein the carbon nanotubes are joined end to end by van der Waals attractive force.

4. The cell culture system of claim 1, further comprising a power supply configured to stimulate the cells with an electric signal.

5. The cell culture system of claim 1, wherein the culture layer comprises two or more stacked carbon nanotube films.

6. The cell culture system of claim 5, wherein the carbon nanotubes in two adjacent carbon nanotube films are substantially parallel to each other.

7. The cell culture system of claim 1, wherein an average diameter of the carbon nanotubes is in a range of about 10.0 nm to about 20.0 nm and a thickness of the culture layer is in a range of about 10.0 nm to 200.0 nm.

8. The cell culture system of claim 1, wherein the carbon nanotubes further comprise of a functional group selected from the group consisting of pyrene, anthracene and carboxylic acid.

9. A cell culture substrate for directing growth of cells, comprising:
a base; and
a culture layer located on the base and comprising at least one carbon nanotube film, the carbon nanotube film comprising a plurality of carbon nanotubes orientated primarily along a same direction.

10. The cell culture substrate of claim 9, wherein the carbon nanotube film comprises of a plurality of successively orientated carbon nanotube segments, the carbon nanotube segments comprise of the plurality of carbon nanotubes.

11. The cell culture substrate of claim 9, wherein the carbon nanotubes are joined end to end by van der Waals attractive force.

12. The cell culture substrate of claim 9, wherein the culture layer comprises two or more stacked carbon nanotube films.

13. The cell culture substrate of claim 12, wherein the carbon nanotubes in two adjacent carbon nanotube films are substantially parallel to each other.

14. The cell culture substrate of claim 11, wherein an average diameter of the carbon nanotubes is in a range of about 10.0 nm to about 20.0 nm and a thickness of the culture layer is in a range of about 10.0 nm to 200.0 nm.

15. The cell culture substrate of claim 11, wherein the carbon nanotubes further comprise of a functional group selected from the group consisting of pyrene, anthracene and carboxylic acid.

16. A culture method for directing the growth of cells, the method comprising the steps of:
providing a substrate; one or more carbon nanotube films drawn from a carbon nanotube array, the carbon nanotube film comprising a plurality of carbon nanotubes orientated primarily along a same direction; and a culture medium comprising at least one growth factor;
placing the carbon nanotube film on the substrate;
covering at least one portion of the carbon nanotube film with the culture medium;
seeding the culture medium with one or more seed cells; and
growing new cells along the carbon nanotubes.

17. The method of claim 16, further comprising a step of stimulating the cells with an electric signal.

18. The method of claim 16, wherein there are at least two of carbon nanotube films that are stacked on each other, the carbon nanotubes in two adjacent carbon nanotube films are substantially parallel to each other.

19. The method of claim 16, further comprising a step of functionalizing the carbon nanotubes.

20. The method of claim 19, wherein the carbon nanotubes are functionalized with a functional group selected from the group consisting of pyrene, anthracene and carboxylic acid.
